SEQUENCE LISTING (32 sequences)

SEQ ID NO: 1 - DNA, 1071 bp, Zea mays
ccggaattcccggggtcgacccacgcgtccggccgcgcgccgtggcgcccgccgacctccagctctccgggtcctacgcctccgacatctacacctacctccgctccctggaggtggatccgcagcggcggtccagatccgattacatcgaggcggtgcaggcggacgtcacggcccacatgcggagcatcctcgtcgactggctcgtcgaggtcgccgaggagtacaagctcgtcgcggacacgctctacctcaccatctcttatgtcgaccgcttcctctccgtcaacgcgctcggccgtgacaagctgcagctccttggcgttgcctccatgctcattgccgcgaagttcgaggagatcagcccgccgcacccggaggacttctgctacatcacagacaacacctacaccaaagaggagctcctcaagatggagagcgacatactcaagcttctcaagttcgagttgggcaatcctacaatcaagaccttcctgagacgtttcataagatctgcccatgaagacaagaagggctccatcttgttaatggaattcttggggagctacctcgctgagctgagtctactagattatggctgcctccggttcttgccatcagtagttgctgcttcagtcatgtttgttgctaggcctgacattgatccaaataccaatccgtggaacacaaagctgcagaagatgactggctacaaagtttctgaactcaaggattgcatcgtagccatacatgacttgcagctcaacaggaaatgtccatcattaacggcaattcgagacaagtacaagcagcacaagttcaaatgcgtgtcattgatcctcgtgcctgtcgtgatccctacttcatactttgaagacttagctgagtagctgctctcggactgtaccgctgtaaggctaacaatctgagctctccttgagctcttagggacaagcagaaaataaccgtttgatgagctttcctctcatttaagtagcgtggtgaaagctatttgtttgaggttctttaggattaaaaaaaaaaaaaaaaaaaaaaaaaaaaaaaaaaaaa

SEQ ID NO: 2 - protein, 295 aa, Zea mays
GIPGVDPRVRPRAVAPADLQLSGSYASDIYTYLRSLEVDPQRRSRSDYIEAVQADVTAHMRSILVDWLVEVAEEYKLVADTLYLTISYVDRFLSVNALGRDKLQLLGVASMLIAAKFEEISPPHPEDFCYITDNTYTKEELLKMESDILKLLKFELGNPTIKTFLRRFIRSAHEDKKGSILLMEFLGSYLAELSLLDYGCLRFLPSVVAASVMFVARPDIDPNTNPWNTKLQKMTGYKVSELKDCIVAIHDLQLNRKCPSLTAIRDKYKQHKFKCVSLILVPVVIPTSYFEDLAE

SEQ ID NO: 3 - DNA, 435 bp, Glycine max
  unsure (1): n = A, C, G or T
nactccatcttcttccatccatttcctctttctcgatctgttccaaattcacttcacacacaggaaagaagatggagactcgcgcngncgcaaagagaaaggcaaatgccgccaccatagtctttgtcgaaaaacaataccccaacaagaggcagcgggttgtgttgggtgaacttcccaatttacaaaaccttattgtctccgaaactcaaaatnngcgcaaagagaagntcctatgtnggaagaatcccaatgagaagaaaccatcacccacaaacaacaacacctttccttcccctcagatcancgaatcttatgattcggatatccacgggtatcttcgtgaaatggagatgcagaataagagaagancaatngttgatacattgaaaaggttagaaaatcgttacccaaccatggagcaatntggtgatt

SEQ ID NO: 4 - protein, 110 aa, Glycine max
  UNSURE (6): Xaa = ANY AMINO ACID
METRAXAKRKANAATIVFVEKQYPNKRQRVVLGELPNLQNLIVSETQNXRKEKXLCXKNPNEKKPSPTNNNTFPSPQIXESYDSDIHGYLREMEMQNKRRXXVDTLKRLE

SEQ ID NO: 5 - DNA, 847 bp, Triticum aestivum
  unsure (584): n = A, C, G or T
cggaacagggagagtggtgttcatgagccattctttcagggaagaaacacaagagataaatctgaaactgctgactcaaacactgggtactatgttggcttaaacgttatagacattgacaaagataatggcaatccacaaatgtgtgcttcctatgctgcagagatatacagaaacctaatggctgcagagcttataaggagacctaaatcaaattacatggagactttgcaaagggatatcacaaagggcatgcgaggaatcctgattgattgggctttgaggttcctggaggaatataaacttttgccagacacactatacctcactgtatatcttattgatcaatttctttctcggaaatatattgaaagacagaaactacaacttcttggaataactagcatgctgattgcctcaaaatatgaagagatctgtgcgcctcgtgttgaagaattttgtttcataactgataacacatatacaaaaaatcaggtgctgaaaatggagtgtgaagtgcttaatgatctggggtttcatctttcagttcccacaatcaaaacgtttctgaggagattccttanagcagcacatgcttctcaaaaaagcccttgggcaactttgggctatctggggcaattatcttgccggagttgacattgaccgattacagttccctgaaattnaacctcaatgggtggaanctcggcgggtccctgcaaaatggcacncgacatcagactgcaanggaatccacctcgagcatanactnaatcaaaanttangtatcaagatgcgtacgnatnnatggaactgacnaggaanacaanggatccccnaaggtata

SEQ ID NO: 6 - protein, 211 aa, Triticum aestivum
  UNSURE (195): Xaa = ANY AMINO ACID
RNRESGVHEPFFQGRNTRDKSETADSNTGYYVGLNVIDIDKDNGNPQMCASYAAEIYRNLMAAELIRRPKSNYMETLQRDITKGMRGILIDWALRFLEEYKLLPDTLYLTVYLIDQFLSRKYIERQKLQLLGITSMLIASKYEEICAPRVEEFCFITDNTYTKNQVLKMECEVLNDLGFHLSVPTIKTFLRRFLXAAHASQKSPWATLGYL

SEQ ID NO: 7 - DNA, 1007 bp, Zea mays
  unsure (924): n = A, C, G or T
gggagggaattccttcctccttttctgttcggcgccgtgctcgcgcgcacccacccgcacgccccagtacccccacgctgcacagtgcacgccgactttcctccgccttgctgctgcaagtccgcaaccactggaggaaaaatcttttccttcacttttcttccctttccccccgcgcatgcacgggctctgattgacgccatgggggacgccgcggcctccacgtccgctcccaccacgcccacctccatcctcatctgcctggaagacggcagcgaccttctcgccgatgccgacgatggcgccggcactgacctcgttgtcgcccgcgacgaacgtctgcttgtcgtggaccaggacgaggagtatgtagcgctgctcctgtccaaggagagcgcgtcaggcggcggcggcccggtggaggaaatggaggactggatgaaggccgcgcgctccggatgcgtccgctggatcatcaagaccacggcgatgttccggttcggcgggaagaccgcttacgtggccgtgaattacctcgatcgcttcctggcgcaacggcgagtcaatagggagcatgcgtggggtctgcagctgctcatggtggcgtgcatgtcgctggcgaccaagctggaggagcaccacgctccgcggctgtcggagttcccgctggacgcgtgcgagttcgcgttcgacagcgcgtccatcctgcggatggagctcctcgtcctgggcaccctcgagtggcggatgatcgccgtcacccccttcccctacatcagctacttcgcggcgcggttccgggagacgagcgccgggcgaatcctcatgcgcgccgtggagtgcgtcttcgcggcgatcaaagtgataagctcggtggagtaacggccgtcgaccatcgccgtggcatccatcctcgtcgcgcgnggccgggaggagactcccgccggcagcctgggangcgctcaaggcgnntcctcgggtcatcgtgcccgcaantagaaaacggg

SEQ ID NO: 8 - protein, 238 aa, Zea mays
  UNSURE (227): Xaa = ANY AMINO ACID
MGDAAASTSAPTTPTSILICLEDGSDLLADADDGAGTDLVVARDERLLVVDQDEEYVALLLSKESASGGGGPVEEMEDWMKAARSGCVRWIIKTTAMFRFGGKTAYVAVNYLDRFLAQRRVNREHAWGLQLLMVACMSLATKLEEHHAPRLSEFPLDACEFAFDSASILRMELLVLGTLEWRMIAVTPFPYISYFAARFRETSAGRILMRAVECVFAAIKVISSVEXRPSTIAVASIL

SEQ ID NO: 9 - DNA, 510 bp, Oryza sativa
  unsure (424): n = A, C, G or T
cttacagcttcctcctcgctttgctggttgcagacgaacgcgggattccggttcagcttgaagacggcgtatgtcgcggtgacgtatctcgatcggttcttggcgcgccggtgtgtcgatagggacaaggagtgggcgctgcagctcctctcggtggcgtgcctgtcgctggcggcgaaggtggaggagcgccggccgccgcggctgccggagttcaagctggacatgtacgactgcgcgtccttgatgcggatggagctcctcgtcctcaccacgctcaagtggcagatgatcaccgagacacccttctcctacctgaactgttcaccgcgaaattccggcacgacgagcggaaggcatcgtcctgcgcgccatcgaatgcatcttcgctcgatcaaagtcatagctcggtgggtacagcatnacgatcgctctagcagnatctatcgtcggaacaaggagacggcgctaattagacgactaagtcgtcgtggctctatggagcactaa

SEQ ID NO: 10 - protein, 181 aa, Oryza sativa
HELTASSSLCWLQTNAGFRFSLKTAYVAVTYLDRFLARRCVDRDKEWALQLLSVACLSLAAKVEERRPPRLPEFKLDMYDCASLMRMELLVLTTLKWQMITETPFSYLNCFTAKFRHDERKAIVLRAIECIFASIKVISSVGYQPSTIALAAILIARNKETAPNLDELSVHRLAPWQLMML

SEQ ID NO: 11 - DNA, 2259 bp, Glycine max
acaacttctcccactcattcatcaacaaccacacacactctctctcccctctctgcaccaaaaccacttctccggcgacatctccggtcaggttccggcaacctcatcggcgaatcggcatgccaacacaatgaatgcggaacctccgctgccgccggcgctcctcatgtcggtttcctgcctctccgactacgacctcctctgcggcgaggactcctccggaatcctctccggagagtcgccggagtgctccttctccgacatcgactcctcacctcctccgccgtcgccgacgacagaggattgttattcgatcgcgagcttcatcgagcacgagcgcaacttcgttccgggattcgagtacctgtcgcggttccaatctcgctccctggacgccaacgccagagaagaatcagttggatggattctcaaggtacacgcgtactatggctttcagcctttgacggcgtacctcgccgtcaactatatggatcggtttttggattctcgccggttgccggaaacaaatgggtggcctctgcaacttgtatctgttgcatgcttgtctttggcagcaaagatggaagaacctcttgttccatctctcttggaccttcagatagaaggtgccaagtacatatttgagccgagaacaattcgtaggatggagctacttgttctcggtgtcttagattggaggctaagatcagtaacaccactttgcttcctcgctttctttgcgtgcaaagtagattcaactggaacttttatccggttccttatttccagggcaacagaaatcatcgtatctaatatccaagaggctagctttcttgcttactggccttcatgcattgctgctgcagccatactcactgcagctaatgaaattcctaattggtctgtggttaagcccgaaaatgctgagtcatggtgcgagggactaagaaaagaaaaagtaatagggtgctaccagttgatgcaagagcttgtgattaacaataaccaacggaaactccccttactaaaagtgttgccgcagctgcgagtaacaactcggacccgaatgaggtcaagtactgtatcatcattctcatcatcctcttcaacctccttctccttgtcttgtaagaggaggaaattaaataaccgtttgtgggtagatgacaaaggaaactccgagtgaagagaaaacgaacaacaataataaaagaagggaagaaaaagagagggaataaggtgggccaagttgtctagaaacctcaacattttttagagggtttttgcaattaaaaaatgacttgagtgagggtgtagattataatagtatatatatgatatatctctatcgtatatactaagagagtttgatgggtttggagtaatttttatttttatgttggtgacttattaatattgagtttgcagaattcacctagggaagagggattttgcgacatgttaccgtgggagaggaaatgagagaagaaagaagtgaaacactgaaccaggggtagaagaatttaatgtgatttgttcttgtaacctgtgattctgaaggaaagaattgagttgcgggctggatttcaaagtttgcattaattacttggtgaaggagatgaaagatggtgggggcaacgctgtagagattgagaagaagaaaaagtagagagaaggtatgaaaaactctggtgattattgaaagttgaaacttagaagtttgaagtgttcaatgttcatatcatggtattcataagtcaagcaaagcttcatttcttggccagcatcactgcttcttcatcatccacgttactactactttgatgggaccctcaacagtaaagaacaattgaagggcaataagttgaagtttggctacaaatcgtggactttttttgttgggtattggcacgtgtgcagtcggttctggtgcgtgccaatgaagtgtgtacgtgtgatttttctttttcttggtttttctttgcgggagctgtttatatattttttcctatttttggccatgagttttggcctaactatacaggactccaatggctggtgtccgcgcgtgtgatggaaacacgtgtatatataggttttaatttaaaaaccttgaatttttttatttgttttcaagagaggagaaccctctttcacataggggtaaaaggtctttggggcctttttcctgtgtgctgtgtattggattgattaatatataatgacaactattaatttcttaaaaaaaaaaaaaaaaaa

SEQ ID NO: 12 - protein, 351 aa, Glycine max
MNAEPPLPPALLMSVSCLSDYDLLCGEDSSGILSGESPECSFSDIDSSPPPPSPTTEDCYSIASFIEHERNFVPGFEYLSRFQSRSLDANAREESVGWILKVHAYYGFQPLTAYLAVNYMDRFLDSRRLPETNGWPLQLVSVACLSLAAKMEEPLVPSLLDLQIEGAKYIFEPRTIRRMELLVLGVLDWRLRSVTPLCFLAFFACKVDSTGTFIRFLISRATEIIVSNIQEASFLAYWPSCIAAAAILTAANEIPNWSVVKPENAESWCEGLRKEKVIGCYQLMQELVINNNQRKLPLLKVLPQLRVTTRTRMRSSTVSSFSSSSSTSFSLSCKRRKLNNRLWVDDKGNSE

SEQ ID NO: 13 - DNA, 1994 bp, Glycine max
gcacgagccggaatcatggattcctcgccggagtgttcctccgacctcgattcctcgccgccgtcggaggcggagtccatcgccggattcatggaagacgagcgcaacttcgtccccggattcgaatacctcaataggttccaatctcgctctctcgacgcctctgccagagaagaatccgttgcatggattctcaaggtgcaggcttattacgcttttcaaccggtcacggcttatctttccgttaactacttggataggttcttgaattctcgaccgttgccgccgaaaacgaatgggtggccactgcaacttctctctgttgcgtgcttgtctttagcagcaaagatggaggaatctctagttccatctcttttggaccttcaggtagaaggtgctaaatacgtatttgaacccaaaacaattagaagaatggagctacttgttctgggcgtgttggattggaggctaagatcggttaccccatttagcttcctcgatttctttgcgtgcaagttagattcaactgggacttttaccgggttcctcatttcacgtgctacacaaattatcttatctaatatacaagaggctagctttcttgcgtattggccatcatgcattgctgcagcagccattctccatgcagcaaatgaaattcctaattggtctctcgttaggcctgagcatgcagagtcatggtgtgaggggttaagaaaggagaaaattatagggtgctaccaattaatgcaagaacttgtgattgacaataaccagaggaaaccccctaaggtgttaccacagctgcgagtgacaatatctcggcccattatgaggtctagtgtctcatccttcttagcatcatcctcttcaccttcatcatcctctttgtcttgtagaaggaggaaattaaataactctttgtgggtagacgatgacaaaggaaactcccaatgaagagaaaaagaacaataatagaggaggaaaaaaagaagaataatgaaataaggtggtggacggtggtccaagttgtccagaaaccgcaaaatttttaagagggtttttttgagtataaaatggactagagagtcgaggtgtacattataatatagactatattttatgagagttgcgagcttttattattttgttggtgggtgttgccattcatcaatggcattgcagattccctagggaaggggattttgcaaagtgtgtgtgggtgtgcgtgagagagaggtgggaaattagatacaaagatgcatttaatggtttggtcgtgcaagcgtggaagaaagaagtgtgtagtttggaattcaaagatgcgcttgttattggtgaaagagaagagaatggtggtgggacattgcttcagagcttggaagaagaaaaaaagcatagtctcagagcagatatcaatagggtattgaaagactttgaagtttgagctgtttcttcttattataatggggtctctaagtcaagctactcatcttggccagcatgcctcgcttcttcagtttacacgttacttttggtagtgtgatgggataccactagagtacaaaaaaagaaatagcaataaggtgaatcttatcacattttgtggacttatgatgatacgtgtgagacgcctctggtgtgtgtctcgcacgtgcctacgtgtgattttttatttttatttatgttttagctatggcgggaaatgctttttatttctttatttcttttttggcttgagctttggcctaactatacaggatccattgcctggtgtccacgcgtgatggaaacacgtgtctatagttttcattttttgttttggattttttcatttgttttcaagagaggagaaccctcttttgttttctttttagtgcctaattggctttgggagaaattggagtaaaggcctttggggccttttcctgagtgctttgtattgaattcattaataatgacactgttaattctataaaaaaaaaaaaaaaaaa

SEQ ID NO: 14 - protein, 318 aa, Glycine max
ARAGIMDSSPECSSDLDSSPPSEAESIAGFMEDERNFVPGFEYLNRFQSRSLDASAREESVAWILKVQAYYAFQPVTAYLSVNYLDRFLNSRPLPPKTNGWPLQLLSVACLSLAAKMEESLVPSLLDLQVEGAKYVFEPKTIRRMELLVLGVLDWRLRSVTPFSFLDFFACKLDSTGTFTGFLISRATQIILSNIQEASFLAYWPSCIAAAAILHAANEIPNWSLVRPEHAESWCEGLRKEKIIGCYQLMQELVIDNNQRKPPKVLPQLRVTISRPIMRSSVSSFLASSSSPSSSSLSCRRRKLNNSLWVDDDKGNSQ

SEQ ID NO: 15 - DNA, 570 bp, Triticum aestivum
  unsure (499): n = A, C, G or T
acagaggttcacctaaaaaaaggctagcagttcttcccaaagagacaacagttctaagaaaagaagcaggagctgcagctggtgagcgtctgtgccctgctgattgcgtgcaagtacgaagagatttgggctccagaggtgaacgacttcatattgttctccgacaacacatatactagggagcagattctgaggatggagaaggcaatcctgaacatgcttgagtggaacctgacagtgcccacaccttacgtcttcctcgtgtgattcgccaaggccgcatcctcctgagataagaagaacggcaaggaggtaaaaggaacaccagattttaacaaatcctcagatgtagtacgtatctccatttgccaaacatgatctattgctgaattctgttctccctggtgtattgtctaaatggagacacgtctttttttcgtggactggcgctctgtagtatggacagaatatgtttgattcagcacacaagagacaggtnatcaacacacagtagnnacagtgtctgtacagccgtatacataacattatacttctcanagaccactttgg

SEQ ID NO: 16 - protein, 75 aa, Triticum aestivum
  UNSURE (68): Xaa = ANY AMINO ACID
KQELQLVSVCALLIACKYEEIWAPEVNDFILFSDNTYTREQILRMEKAILNMLEWNLTVPTPYVFLVXFAKAASS

SEQ ID NO: 17 - DNA, 1932 bp, Zea mays
  unsure (8): n = A, C, G or T
gccacaantgcaccgcagacsgcacntsggcctccctcctccgtccgtccgtcctcttccttgtgccttgtcctcctccactcgccactgccgcattctgcccaagtcccaaacacgcgcaccagccacccagcactccagccgccagaccagagtctncggcgccgccgtcgcacgacaggagagggagagatacgcgggctttgacttgccgccggtgcgtccgtgcgtgcctggtgggaatagtgggagacgccggtacagtacaggagccatggcgccgagctgctacgacgcggcagcgtccatgctcctctgcgccgaggagcacagcagcatcctgtggtacgaggaggaggaggaggagctggaggcggtcgggagaaggagcggccggtcgccgggctacggggacgacttcggcgcggacttgttcccgccgcagtcggaggaatgcgtggccggtctggtggagcgggaacgggaccacatgccggggccgtgctacggcgacaggctgcgcggcggcggcggctgtctctgcgtccgccgggaggccgtcgactggatttggaaggcttacacgcaccacaggttccgccctctcactgcctacttggcagtgaactacctcgatcgcttcctctcgctgtctgaggtgccggactgcaaggactggatgacgcagctcctcgcggtggcgtgcgtttctctggccgccaagatggaggaaaccgccgtcccgcagtgcctggaccttcaggaggtcggagacgcgcggtacgtgttcgaggcgaagacggtccagaggatggagctcctggttctaacaaccctcaactggaggatgcatgccgtgacgccgttctcctacgtggattacttcctgaacaagctcaacaacggcggcagcacggcgccgaggagctgctggctcttgcagtccgcggagcttatcttgcgtgcggccagaggaaccggctgcgtcgggttcaggccgtccgagatcgccgccgcggttgcagccgccgtggccggagacgtggacgacgcggacggcgtcgagaacgcctgctgcgctcacgtagataaggagcgggtgttgcggtgccaggaagcgatcgggtccatggcgtcctcggcggccattgacgacgctaccgtgccaccgaaatctgcgagacgcaggagctcccccgtgcccgtgccgcagagccctgtgggggtgctggacgcggctccctgcctcagctacaggagcgaagaggcagcgactgcgactgcgactgcgacttctgctgcctcacatggggcccctggctcttcaagctcgtcctcgacctccccggtgaccagcaaaaggaggaaactcgccagccgatgtgatggatcgtgcagtgaccggtcaaagcgcgcgcccgcccaatggaccaaagagtgaattgactagggcgtctgcttgctttctgatcaaagagtgcattgagaggcggcaaaatggaggaataaaggggattttggcatgacgagggcaaaggagttgatgaataaagacgcgacgaggtggacaacacctaaattgccgatcttttctttgcaaggggagtaggggacctgcttgggcctgggggagggtagtagacagcccagcacaaaaacctggttggtgtgccacgaccacaatgggcgcgccagccatggctttgtaggaaacacaagggcgctagaggagatccgatgggatgactcagaataaagatagtggagggaccagaccgtatgcagtatgtgcaacaactaggcactggcatgcttatgctcaagtaatctgataacttgaatgttgtgtatccgacaaactgcttctgtgaagagagaaatgcaggtagacgatgaatgtatgtgaaaaaaaaaaaaaaaaaaaaaaaaaac

SEQ ID NO: 18 - protein, 388 aa, Zea mays
MAPSCYDAAASMLLCAEEHSSILWYEEEEEELEAVGRRSGRSPGYGDDFGADLFPPQSEECVAGLVERERDHMPGPCYGDRLRGGGGCLCVRREAVDWIWKAYTHHRFRPLTAYLAVNYLDRFLSLSEVPDCKDWMTQLLAVACVSLAAKMEETAVPQCLDLQEVGDARYVFEAKTVQRMELLVLTTLNWRMHAVTPFSYVDYFLNKLNNGGSTAPRSCWLLQSAELILRAARGTGCVGFRPSEIAAAVAAAVAGDVDDADGVENACCAHVDKERVLRCQEAIGSMASSAAIDDATVPPKSARRRSSPVPVPQSPVGVLDAAPCLSYRSEEAATATATATSAASHGAPGSSSSSSTSPVTSKRRKLASRCDGSCSDRSKRAPAQWTKE

SEQ ID NO: 19 - DNA, 481 bp, Oryza sativa
  unsure (88): n = A, C, G or T
cttacatgtaagctcgtgccgaattcggcacgagcttacacgagcgcaacccatgggcgccgcggctgctcgccatcagctgcctcancctcgccgccaagatgcagcgcgccgccgccatctccgccgncgacatccagaggggcgaggagttcatgttcgacgaggcgaaaatccagcgcatggagcagatggtgctcaacgcgctggagtggcggacgcgctccgtcacgccgctcgccttcctcggnttctttctctccgcgtggttcccgcaagccgcggcacccggcgctgctcgatgccatcanggccgcgccgtcgagctcctcctccgcgtctaagccgggangtgaacnatggtgggagttctcccccttcggtggccggccgncgcggntctcctcncngccgncgganaaggcntccggnngcccaaactccttcncttccaaanctgnnggccccgntttgncccctt

SEQ ID NO: 20 - protein, 110 aa, Oryza sativa
  UNSURE (26): Xaa = ANY AMINO ACID
ARAEFGTSLHERNPWAPRLLAISCLXLAAKMQRAAAISAXDIQRGEEFMFDEAKIQRMEQMVLNALEWRTRSVTPLAFLGFFLSAWFPQAAAPGAARCHXGRAVELLLRV

SEQ ID NO: 21 - DNA, 789 bp, Triticum aestivum
cacctgagggcgactcgagggtgccctcgccccgtccgccgtgaccacccctcttcggatctcaccgcctcgaccaaaatgtgatttgaggcaaattctgcgtttgaggcaaggacaataaaagtgatggagcttttggtcttcagcaccttgaaatggaggatgcaagctgttactgcttgctcgtttattgactacttcctttgcaaattcaatgatcatgacacaccctccatgcttgcattctcctgctcaactgacctcatcctgagcacaactaagtgagctgattttttggtgttcagacattcagagattgctggaagtgttgcacttccttcatttggggagcacaagacttcagttgtcgaaatggctacaactaattgcaagtatataaacaagggagtgtgatgtgacaggaaagatcctgatgaagtgcttcctttatggaatgcctatctgaagtttggactaagagacatgctttaattggcttagtaaaaaatacttgctaaagagaaataagattcaaagtagatgtttttattgtagattaggatatgtgtgttctgccaccggttcgacttctcatattagaaggcaagcagttagttcatatcttactactttgcactattgtagatggatggtgagggattgagaggctactactattaatgtgcgtaaactttgcatctttagctctctaaatgaaaccggtgatggttaacctgaaaaaaaaaaaaaaaaaaaaaaaaaaaaaaaaaaaaaaaaaaaaaaaaaaaa

SEQ ID NO: 22 - protein, 163 aa, Triticum aestivum
  UNSURE (28): Xaa = ANY AMINO ACID
HLRATRGCPRPVRRDHPSSDLTASTKMXFEANSAFEARTIKVMELLVFSTLKWRMQAVTACSFIDYFLCKFNDHDTPSMLAFSCSTDLILSTTKXADFLVFRHSEIAGSVALPSFGEHKTSVVEMATTNCKYINKGVXCDRKDPDEVLPLWNAYLKFGLRDML

SEQ ID NO: 23 - DNA, 1132 bp, Zea mays
  unsure (441): n = A, C, G or T
aacagaattcggcacgagccgcggtcggctgggtttcacgcgccgcggcgcggctaggcttctccgcgctcaccgccgcgctcgccgccgcctacctcgaccgctgcttcctccccgggggcgcgctccggctcggcgaccagccctggatggcgcgcctagccgccgtcacctgcttcgcgctcgccgccaaggtcgaggagacgcgcgtgccgccgctcctcgacctccagctctacgccgccgctgacgccgcggatccgtacgtattcgaggccaagacggtgcgccggatggagctgctcgtgctctccgcgcttgggtggcggatgcaccctgtcacgcccttctcctacctccagcccgtcctcgccgacgctgcgacgcgcctgcgtagctgcgagggcgtcctgctcgcggtcatggccgactggaggtggcctcggcaccggccttcggcgtgggccgccgccgcgttgctgatcacagccgccgccggcgacggcggcgacggcgacggcgacacggagctcctggcgctcatcaatgcccccgaggacaagaccgccgagtgtgccaagatcatctccgaggtgacgggcatgagcttcctcgcctgcgatgtcggcgtgagcgccggaaataagcgtaagcacgcggcggcgcagttgtactcgccgccgccgagcccgagcggcgtgatcggcgcgctgtcctgcttcagctgcgagagctcgacgtccgccaccgctatggctgcggcggtcggcccgtgggcgccgtcggcgtccgtgtccgtgtcgtcctctccagagccaccaggtcgggcccccaagcgcgcagcggcggcgtcggcgtcggcgtcggcgtcagccggggtcgcgccaccggtccaggtcccgcatcagctaccccccgacgaggagagccgcgacgcctggccgtccacctgcgccgcgtgacgcaccgtgccggaaacggtgcctatggcgagaccgccgttcggtggcggtggagaatggagaacaaggagcatcattggctcgcgtcggtgagcaggagaacgaactattttgcccattgccgtgacagatggggggtgttcactgcgtggagccgcgctgancaatga

SEQ ID NO: 24 - protein, 318 aa, Zea mays
NSARAAVGWVSRAAARLGFSALTAALAAAYLDRCFLPGGALRLGDQPWMARLAAVTCFALAAKVEETRVPPLLDLQLYAAADAADPYVFEAKTVRRMELLVLSALGWRMHPVTPFSYLQPVLADAATRLRSCEGVLLAVMADWRWPRHRPSAWAAAALLITAAAGDGGDGDGDTELLALINAPEDKTAECAKIISEVTGMSFLACDVGVSAGNKRKHAAAQLYSPPPSPSGVIGALSCFSCESSTSATAMAAAVGPWAPSASVSVSSSPEPPGRAPKRAAAASASASASAGVAPPVQVPHQLPPDEESRDAWPSTCAA

SEQ ID NO: 25 - DNA, 674 bp, Glycine max
  unsure (527): n = A, C, G or T
cactcactcaccccttcctttctaactcctcaaattgtgtgttctgagaatggaaatgcctccttctccatcggggcattccgcactctccatcccataaaagtcccagatccaagatggcttaccaccatcaaaaatcccttttggacaccctatactgctccgaagagcattggataggggaaggtgaatttgaccaagcagaggaggagtacggtaacagtaatagcaatagtagcagcaccttagtaaacaactcccctgagtcctcccctcatttgttgctcgaaagcgacatgttttgggacgaacaagagttggcatcgctgttggagaaagaacaacacaacccactaagcacttgctgtctccaaagcaaccctgccttggagggtgctcgcatagaagccgtggagtggattctcaaagtaaacgcccactactccttctctgccctcaccgctgttcttgctgtcaactactttgaccgttttctcttcagcttccgctttcagaatgacattaanccatggatgactcggggtcgctgccgtcgcttgnctctccctcgctgccaaagtgggcgagacacacgttccctttcttatttgacccttcaacaaagtgggaggaggagtanatnctttgttccaagccaaagacgattaaaaaag

SEQ ID NO: 26 - protein, 186 aa, Glycine max
  UNSURE (137): Xaa = ANY AMINO ACID
MAYHHQKSLLDTLYCSEEHWIGEGEFDQAEEEYGNSNSNSSSTLVNNSPESSPHLLLESDMFWDEQELASLLEKEQHNPLSTCCLQSNPALEGARIEAVEWILKVNAHYSFSALTAVLAVNYFDRFLFSFRFQNDIXPWMTRGRCRRLXLPRCQSGRDTRSLSYLTLQQSGRRSXXFVPSQRRLKK

SEQ ID NO: 27 - DNA, 554 bp, Glycine max
ctccctttcacctttcttcatagcctaccacttttctgctttcatctactctcacttctcttcacacactgagacacacagagagagaaaaataaagggtgtgatgggtgtcttactgagtgttttctttttataatgaacaaagaactgcacaccctcttcttcaccgaagaagaagatggcaattcagcaccacaatgaccaactagagcataatgaaaatgtctcatctgtccttgatgccctttactgtgacgaaggaaagtgggaagaggaagaggaggagaaagaagaagaagaagatgaaggtgaaaatgaaagtgaagtgacaacaaacactgcaacttgtcttttccctctgctcttgttggagcaagacttgttctgggaagatgaggaactaaactctatcttttccaaagagaaggttcaacatgaagaagcctatggtataacaatctgaacagtgatgtgtataacaacaacaacaatactagtatataatgtgatttggctcttgctcttcagctcgtcggagcgtgatgatgctgaat

SEQ ID NO: 28 - protein, 94 aa, Glycine max
MAIQHHNDQLEHNENVSSVLDALYCDEGKWEEEEEEKEEEEDEGENESEVTTNTATCLFPLLLLEQDLFWEDEELNSIFSKEKVQHEEAYGITI

SEQ ID NO: 29 - protein, 372 aa, Catharanthus roseus
MADKENCIRVTRLAKKRAVEAMAASEQQRPSKKRVVLGELKNLSSNISSIQTYDFSSGPQKQQKNKNKRKAKESLGFEVKEKKVEEAGIDVFSQSDDPQMCGAYVSDIYEYLHKMEMETKRRPLPDYLDKVQKDVTANMRGVLIDWLVEVAEEYKLLPDTLYLTVSYIDRFLSMNALSRQKLQLLGVSSMLIASKYEEISPPHVEDFCYITDNTYKKEEVVKMEADVLKFLKFEMGNPTIKTFLRRLTRVVQDGDKNPNLQFEFLGYYLAELSLLDYGCVKFLPSLIASSVIFLSRFTLQPKVHPWNSLLQHNSGYKPADLKECVLIIHDLQLSKRGSSLVAVRDKYKQHKFKCVSTLTAPPSIPDEFFEDI

SEQ ID NO: 30 - protein, 335 aa, Arabidopsis thaliana
MRSYRFSDYLHMSVSFSNDMDLFCGEDSGVFSGESTVDFSSSEVDSWPGDSIACFIEDERHFVPGHDYLSRFQTRSLDASAREDSVAWILKVQAYYNFQPLTAYLAVNYMDRFLYARRLPETSGWPMQLLAVACLSLAAKMEEILVPSLFDFQVAGVKYLFEAKTIKRMELLVLSVLDWRLRSVTPFDFISFFAYKIDPSGTFLGFFISHATEIILSNIKEASFLEYWPSSIAAAAILCVANELPSLSSVVNPHESPETWCDGLSKEKIVRCYRLMKAMAIENNRLNTPKVIAKLRVSVRASSTLTRPSDESSSPCKRRKLSGYSWVGDETSTSN

SEQ ID NO: 31 - protein, 354 aa, Nicotiana tabacum
MAADNIYDFVASNLLCTETKSLCFDDVDSLTISQQNIETKSKDLSFNNGIRSEPLIDLPSLSEECLSFMVQREMEFLPKDDYVERLRSGDLDLSVRKEALDWILKAHMHYGFGELSFCLSINYLDRFLSLYELPRSKTWTVQLLAVACLSLAAKMEEINVPLTVDLQVGDPKFVFEGKTIQRMELLVLSTLKWRMQAYTPYTFIDYFMRKMNGDQIPSRPLISGSMQLILSIIRSIDFLEFRSSEIAASVAMSVSGEIQAKDIDKAMPCFFIHLDKGRVQKCVELIQDLTTATITTAAAASLVPQSPIGVLEAAACLSYKSGDERTVGSCTTSSHTKRRKLDTSSLEHGTSEKL

SEQ ID NO: 32 - protein, 373 aa, Nicotiana tabacum
MAIEHNEQQELSQSFLLDALYCEEEEEKWGDLVDDETIITPLSSEVTTTTTTTTKPNSLLPLLLLEQDLFWEDEELLSLFSKEKETHCWFNSFQDDSLLCSARVDSVEWILKVNGYYGFSALTAVLAINYFDRFLTSLHYQKDKPWMIQLAAVTCLSLAAKVEETQVPLLLDFQVEDAKYVFEAKTIQRMELLVLSSLKWRMNPVTPLSFLDHIIRRLGLRNNIHWEFLRRCENLLLSIMADCRFVRYMPSVLATAIMLHVIHQVEPCNSVDYQNQLLGVLKINKEKVNNCFELISEVCSKPISHKRKYENPSHSPSGVIDPIYSSESSNDSWDLESTSSYFPVFKKSRVQEQQMKLASSISRVFVEAVGSPH

(19) United States
(12) Patent Application Publication
(10) Pub. No.: US 2005/0148765 A1
(43) Pub. Date: Jul. 7, 2005

(54) Plant cell cyclin genes

(76) Inventors: Rebecca E. Cahoon, Webster Groves, MO (US); Karlene H. Butler, Newark, DE (US); William James Gordon-Kamm, Urbandale, IA (US); Keith S. Lowe, Johnston, IA (US); Yumin Tao, Ames, IA (US); Emil M. Orozco JR., Cochranville, PA (US); Theodore M. Klein, Wilmington, DE (US); Joan T. Odell, Unionville, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/814,492

(22) Filed: Mar. 31, 2004

Related U.S. Application Data
(62) Division of application No. 09/665,308, filed on Sep. 19, 2000, now abandoned.
Application No. 09/665,308 is a continuation of application No. PCT/US99/06047, filed on Mar. 19, 1999.
(60) Provisional application No. 60/078,948, filed on Mar. 23, 1998.

(51) Int. Cl. C07H 21/04; C07K 14/415; A01H 1/00; C12N 15/82; C12Q 1/68; C12N 5/04

(52) U.S. Cl. 536/23.6; 530/370; 435/69.1; 435/468; 435/419; 800/278

(57) ABSTRACT

This invention relates to an isolated nucleic acid fragment encoding a cyclin protein. The invention also relates to the construction of a chimeric gene encoding all or a portion of the cyclin protein, in sense or antisense orientation, wherein expression of the chimeric gene results in production of altered levels of the cyclin protein in a transformed host cell.

This application claims the benefit of U.S. Provisional Application No. 60/078,948, filed Mar. 23, 1998.

### FIELD OF THE INVENTION

This invention is in the field of plant molecular biology. More specifically, this invention pertains to nucleic acid fragments encoding cyclin proteins in plants and seeds.

### BACKGROUND OF THE INVENTION

Cells divide by duplicating their chromosomes and segregating one copy of each duplicated chromosome, as well as providing essential organelles, to each of two daughter cells. Regulation of cell division is critical for the normal development of multicellular organisms. A cell that is destined to grow and divide must pass through specific phases of a cell cycle: G₁, S (period of DNA synthesis), G₂, and M (mitosis). Studies have shown that cell division is controlled via the regulation of two critical events during the cell cycle: initiation of DNA synthesis and the initiation of mitosis. Several kinase proteins control cell cycle progression through these events. These protein kinases are heterodimeric proteins, having a cyclin-dependent kinase (Cdks) subunit and a cyclin subunit that provides the regulatory specificity to the heterodimeric protein. These heterodimeric proteins regulate cell cycle by interacting with proteins involved in the initiation of DNA synthesis and mitosis and phosphorylating them at specific regulatory sites, activating some and inactivating others. The cyclin subunit concentration varies in phase with cell cycle while the concentration of the Cdks remain relatively constant throughout the cell cycle.

In eukaryotic cells several different cyclin proteins have been identified that regulate cell cycle. Cyclins D (delta) and E appear to function during G₁ phase to regulate progression to S phase (Soni, B. et al. (1995) Plant Cell 7(1):85-103; Sorrell, D. A. et al. (1999) Plant Physiol. 199:343-351). Cyclin A functions during S and G₂ phases to regulate DNA synthesis and cell cycle progression into mitosis and Cyclin B functions only during G₂ phase to control cell cycle entry into mitosis (Kouchi, H. et al. (1995) Plant Cell 7(8):143-1155). Because the cyclin subunit provides specificity for controlling the cell cycle they are obvious targets for manipulating cell-cycle regulation in eukaryotes. There is a great deal of interest in identifying the genes that encode cyclins in plants. These genes may be used to express cyclins in plant cells to enhance cell tissue culture growth. Accordingly, the availability of nucleic acid sequences encoding all or a portion of cyclins would facilitate studies to better understand cell cycle in plants, provide genetic tools to enhance cell growth in tissue culture, increase the efficiency of gene transfer and help provide more stable transformations. Cyclins may also provide targets to facilitate design and/or identification of inhibitors of cyclins that may be useful as herbicides.

### SUMMARY OF THE INVENTION

The instant invention relates to isolated nucleic acid fragments encoding cyclin proteins. Specifically, this invention concerns an isolated nucleic acid fragment encoding a cyclin A, cyclin delta-1, cyclin delta-2 or cyclin delta-3. In addition, this invention relates to a nucleic acid fragment that is complementary to the nucleic acid fragment encoding cyclin A, cyclin delta-1, cyclin delta-2 or cyclin delta-3.

An additional embodiment of the instant invention pertains to a polypeptide encoding all or a substantial portion of a cyclin protein selected from the group consisting of cyclin A, cyclin delta-1, cyclin delta-2 or cyclin delta-3.

In another embodiment, the instant invention relates to a chimeric gene encoding a cyclin A, cyclin delta-1, cyclin delta-2 or cyclin delta-3, or to a chimeric gene that comprises a nucleic acid fragment that is complementary to a nucleic acid fragment encoding a cyclin A, cyclin delta-1, cyclin delta-2 or cyclin delta-3, operably linked to suitable regulatory sequences, wherein expression of the chimeric gene results in production of levels of the encoded protein in a transformed host cell that is altered (i.e., increased or decreased) from the level produced in an untransformed host cell.

In a further embodiment, the instant invention concerns a transformed host cell comprising in its genome a chimeric gene encoding a cyclin A, cyclin delta-1, cyclin delta-2 or cyclin delta-3, operably linked to suitable regulatory sequences. Expression of the chimeric gene results in production of altered levels of the encoded protein in the transformed host cell. The transformed host cell can be of eukaryotic or prokaryotic origin, and include cells derived from higher plants and microorganisms. The invention also includes transformed plants that arise from transformed host cells of higher plants, and seeds derived from such transformed plants.

An additional embodiment of the instant invention concerns a method of altering the level of expression of a cyclin A, cyclin delta-1, cyclin delta-2 or cyclin delta-3 in a transformed host cell comprising: a) transforming a host cell with a chimeric gene comprising a nucleic acid fragment encoding a cyclin A, cyclin delta-1, cyclin delta-2 or cyclin delta-3; and b) growing the transformed host cell under conditions that are suitable for expression of the chimeric gene wherein expression of the chimeric gene results in production of altered levels of cyclin A, cyclin delta-1, cyclin delta-2 or cyclin delta-3 in the transformed host cell.

An addition embodiment of the instant invention concerns a method for obtaining a nucleic acid fragment encoding all or a substantial portion of an amino acid sequence encoding a cyclin A, cyclin delta-1, cyclin delta-2 or cyclin delta-3.

A further embodiment of the instant invention is a method for evaluating at least one compound for its ability to inhibit the activity of a cyclin A, cyclin delta-1, cyclin delta-2 or cyclin delta-3, the method comprising the steps of: (a) transforming a host cell with a chimeric gene comprising a nucleic acid fragment encoding a cyclin A, cyclin delta-1, cyclin delta-2 or cyclin delta-3, operably linked to suitable regulatory sequences; (b) growing the transformed host cell under conditions that are suitable for expression of the chimeric gene wherein expression of the chimeric gene results in production of cyclin A, cyclin delta-1, cyclin delta-2 or cyclin delta-3 in the transformed host cell; (c) optionally purifying the cyclin A, cyclin delta-1, cyclin delta-2 or cyclin delta-3 expressed by the transformed host cell; (d) treating the cyclin A, cyclin delta-1, cyclin delta-2 or cyclin delta-3 with a compound to be tested; and (e) comparing the activity of the cyclin A, cyclin delta-1, cyclin delta-2 or cyclin delta-3 that has been treated with a test compound to the activity of an untreated cyclin A, cyclin delta-1, cyclin delta-2 or cyclin delta-3, thereby selecting compounds with potential for inhibitory activity.

### BRIEF DESCRIPTION OF THE DRAWINGS AND SEQUENCE DESCRIPTIONS

The invention can be more fully understood from the following detailed description and the accompanying drawings and Sequence Listing which form a part of this application.

FIG. 1 shows a comparison of the amino acid sequence set forth in SEQ ID NO:2 and the Catharanthus roseus sequence, SEQ ID NO:29.

FIG. 2 shows a comparison of the amino acid sequences set forth in SEQ ID NOs:8, 10, 12 and 14 and the A. thaliana cyclin delta-1 sequence, SEQ ID NO:30.

FIG. 3 shows a comparison of the amino acid sequences set forth in SEQ ID NOs:18 and 22 and the Nicotina tabacum sequence, SEQ ID NO:31.

FIG. 4 shows a comparison of the amino acid sequences set forth in SEQ ID NO:24 and the Nicotiana tabacum sequence, SEQ ID NO:32.

The following sequence descriptions and Sequence Listing attached hereto comply with the rules governing nucleotide and/or amino acid sequence disclosures in patent applications as set forth in 37 C.F.R. §1.821-1.825.

SEQ ID NO:1 is the nucleotide sequence comprising a contig assembled from the cDNA inserts in clones p0072.comf188rb and cen3n.pk0208.h3 encoding a portion of a corn cyclin A protein.

SEQ ID NO:2 is the deduced amino acid sequence of a portion of a cyclin A protein derived from the nucleotide sequence of SEQ ID NO:1.

SEQ ID NO:3 is the nucleotide sequence comprising a portion of the cDNA insert in clone srm.pk0017.h9 encoding a portion of a soybean cyclin A protein.

SEQ ID NO:4 is the deduced amino acid sequence of a portion of a cyclin A protein derived from the nucleotide sequence of SEQ ID NO:3.

SEQ ID NO:5 is the nucleotide sequence comprising a contig assembled from the cDNA inserts in clones w1mk1.pk0009.b7 and wr1.pk0093.f11 encoding a portion of a wheat cyclin A protein.

SEQ ID NO:6 is the deduced amino acid sequence of a portion of a cyclin A protein derived from the nucleotide sequence of SEQ ID NO:1.

SEQ ID NO:7 is the nucleotide sequence comprising a contig assembled from the cDNA inserts in clones p0128.cpiad46rb, p0116.cesaf50r and p0098.cdfae90r encoding a portion of a corn cyclin delta-1 protein.

SEQ ID NO:8 is the deduced amino acid sequence of a portion of a cyclin delta-1 protein derived from the nucleotide sequence of SEQ ID NO:7.

SEQ ID NO:9 is the nucleotide sequence comprising a portion of the cDNA insert in clone r10n.pk0031.e6 encoding a portion of a rice cyclin delta-1 protein.

SEQ ID NO:10 is the deduced amino acid sequence of a portion of a cyclin delta-1 protein derived from the nucleotide sequence of SEQ ID NO:9.

SEQ ID NO:11 is the nucleotide sequence comprising a contig assembled from the cDNA inserts in clones sah1c.pk003.i7 and sr1.pk0001.g5 encoding an entire soybean cyclin delta-1 protein.

SEQ ID NO:12 is the deduced amino acid sequence of a cyclin delta-1 protein derived from the nucleotide sequence of SEQ ID NO:11.

SEQ ID NO:13 is the nucleotide sequence comprising a portion of the cDNA insert in clone se6.pk0028.f11 encoding a portion of a soybean cyclin delta-1 protein.

SEQ ID NO:14 is the deduced amino acid sequence of a portion of a cyclin delta-1 protein derived from the nucleotide sequence of SEQ ID NO:13.

SEQ ID NO:15 is the nucleotide sequence comprising a portion of the cDNA insert in clone w1e1n.pk0036.e2 encoding a portion of a wheat cyclin delta-1 protein.

SEQ ID NO:16 is the deduced amino acid sequence of a portion of a cyclin delta-1 protein derived from the nucleotide sequence of SEQ ID NO:15.

SEQ ID NO:17 is the nucleotide sequence comprising a portion of the cDNA insert in clone ceb5.pk0049.h5 encoding a portion of a corn cyclin delta-2 protein.

SEQ ID NO:18 is the deduced amino acid sequence of a portion of a cyclin delta-2 protein derived from the nucleotide sequence of SEQ ID NO:17.

SEQ ID NO:19 is the nucleotide sequence comprising a portion of the cDNA insert in clone r10n.pk091.m14 encoding a portion of a rice cyclin delta-2 protein.

SEQ ID NO:20 is the deduced amino acid sequence of a portion of a cyclin delta-2 protein derived from the nucleotide sequence of SEQ ID NO:19.

SEQ ID NO:21 is the nucleotide sequence comprising a portion of the cDNA insert in clone wre1n.pk0104.c1 encoding a portion of a wheat cyclin delta-2 protein.

SEQ ID NO:22 is the deduced amino acid sequence of a portion of a cyclin delta-2 protein derived from the nucleotide sequence of SEQ ID NO:21.

SEQ ID NO:23 is the nucleotide sequence comprising a portion of the cDNA insert in clone cr1n.pk0185.g7 encoding a portion of a corn cyclin delta-3 protein.

SEQ ID NO:24 is the deduced amino acid sequence of a portion of a cyclin delta-3 protein derived from the nucleotide sequence of SEQ ID NO:23.

SEQ ID NO:25 is the nucleotide sequence comprising a contig assembled from the cDNA inserts in clones ses9c.pk002.h24, sr1.pk00.d11, scb1c.pk002.c13 encoding a portion of a soybean cyclin delta-3 protein.

SEQ ID NO:26 is the deduced amino acid sequence of a portion of a cyclin delta-3 protein derived from the nucleotide sequence of SEQ ID NO:25.

SEQ ID NO:27 is the nucleotide sequence comprising a contig assembled from the cDNA inserts in clones sf11.pk0001.a8 and sre.pk0035.b5 encoding a portion of a soybean cyclin delta-3 protein.

SEQ ID NO:28 is the deduced amino acid sequence of a portion of a cyclin delta-3 protein derived from the nucleotide sequence of SEQ ID NO:27.

SEQ ID NO:29 is the amino acid sequence of a Catharanthus roseus (NCBI Identifier No. gi 2190259) cyclin A protein.

SEQ ID NO:30 is the amino acid sequence of an Arabidopsis thaliana (NCBI Identifier No. gi 3915635) cyclin delta-1 protein.

SEQ ID NO:31 is the amino acid sequence of a Nicotiana tabacum (NCBI Identifier No. gi 4160298) cyclin delta-2 protein.

SEQ ID NO:32 is the amino acid sequence of a Nicotiana tabacum (NCBI Identifier No. gi 4160300) cyclin delta-3 protein.

The Sequence Listing contains the one letter code for nucleotide sequence characters and the three letter codes for amino acids as defined in conformity with the IUPAC-IUBMB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

In the context of this disclosure, a number of terms shall be utilized. As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA. As used herein, “contig” refers to an assemblage of overlapping nucleic acid sequences to form one contiguous nucleotide sequence. For example, several DNA sequences can be compared and aligned to identify common or overlapping regions. The individual sequences can then be assembled into a single contiguous nucleotide sequence.

As used herein, “substantially similar” refers to nucleic acid fragments wherein changes in one or more nucleotide bases results in substitution of one or more amino acids, but do not affect the functional properties of the protein encoded by the DNA sequence.

“Substantially similar” also refers to nucleic acid fragments wherein changes in one or more nucleotide bases does not affect the ability of the nucleic acid fragment to mediate alteration of gene expression by antisense or co-suppression technology. “Substantially similar” also refers to modifications of the nucleic acid fragments of the instant invention such as deletion or insertion of one or more nucleotides that do not substantially affect the functional properties of the resulting transcript vis-à-vis the ability to mediate alteration of gene expression by antisense or co-suppression technology or alteration of the functional properties of the resulting protein molecule. It is therefore understood that the invention encompasses more than the specific exemplary sequences.

For example, it is well known in the art that antisense suppression and co-suppression of gene expression may be accomplished using nucleic acid fragments representing less than the entire coding region of a gene, and by nucleic acid fragments that do not share 100% sequence identity with the gene to be suppressed. Moreover, alterations in a gene which result in the production of a chemically equivalent amino acid at a given site, but do not effect the functional properties of the encoded protein, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products.

Moreover, substantially similar nucleic acid fragments may also be characterized by their ability to hybridize, under stringent conditions (0.1×SSC, 0.1% SDS, 65° C.), with the nucleic acid fragments disclosed herein.

Substantially similar nucleic acid fragments of the instant invention may also be characterized by the percent similarity of the amino acid sequences that they encode to the amino acid sequences disclosed herein, as determined by algorithms commonly employed by those skilled in this art. Preferred are those nucleic acid fragments whose nucleotide sequences encode amino acid sequences that are 80% similar to the amino acid sequences reported herein. More preferred nucleic acid fragments encode amino acid sequences that are 90% similar to the amino acid sequences reported herein. Most preferred are nucleic acid fragments that encode amino acid sequences that are 95% similar to the amino acid sequences reported herein. Sequence alignments and percent similarity calculations were performed using the Megalign program of the LASARGENE bioinformatics computing suite (DNASTAR Inc., Madison, Wis.). Multiple alignment of the sequences was performed using the Clustal method of alignment (Higgins, D. G. and Sharp, P. M. (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to afford specific identification and/or isolation of a nucleic acid fragment comprising the sequence. The instant specification teaches partial or complete amino acid and nucleotide sequences encoding one or more particular plant proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

“Codon degeneracy” refers to divergence in the genetic code permitting variation of the nucleotide sequence without effecting the amino acid sequence of an encoded polypeptide. Accordingly, the instant invention relates to any nucleic acid fragment that encodes all or a substantial portion of the amino acid sequence encoding the cyclin A, cyclin delta-1, cyclin delta-2 or cyclin delta-3 proteins as set forth in SEQ ID NOs:2, 4, 6, 8, 10, 12, 14, 16, 18, 20, 22, 24, 26 and 28. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available. “Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a DNA sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. New promoters of various types useful in plant cells are constantly being discovered; numerous examples may be found in the compilation by Okamuro and Goldberg, (1989) Biochemistry of Plants 15:1-82. It is further recognized that since in most cases the exact boundaries of regulatory sequences have not been completely defined, DNA fragments of different lengths may have identical promoter activity.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, MRNA stability or translation efficiency. Examples of translation leader sequences have been described (Turner, R. and Foster, G. D. (1995) Molecular Biotechnology 3:225).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from MRNA. “Sense” RNA refers to RNA transcript that includes the MRNA and so can be translated into protein by the cell. “Antisense RNA” refers to a RNA transcript that is complementary to all or part of a target primary transcript or MRNA and that blocks the expression of a target gene (U.S. Pat. No. 5,107,065, incorporated herein by reference). The complementarity of an antisense RNA may be with any part of the specific gene transcript, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (MRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020, incorporated herein by reference).

“Altered levels” refers to the production of gene product(s) in transgenic organisms in amounts or proportions that differ from that of normal or non-transformed organisms.

“Mature” protein refers to a post-translationally processed polypeptide; i.e., one from which any pre- or propeptides present in the primary translation product have been removed. “Precursor” protein refers to the primary product of translation of MRNA; i.e., with pre- and propeptides still present. Pre- and propeptides may be but are not limited to intracellular localization signals.

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels, J. J., (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632).

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050, incorporated herein by reference).

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Maniatis”).

Nucleic acid fragments encoding at least a portion of several cyclin proteins have been isolated and identified by comparison of random plant cDNA sequences to public databases containing nucleotide and protein sequences using the BLAST algorithms well known to those skilled in the art. Table 1 lists the proteins that are described herein, and the designation of the cDNA clones that comprise the nucleic acid fragments encoding these proteins.

TABLE 1
Cyclin Proteins

| Enzyme | Clone | Plant |
|---|---|---|
| Cyclin A | cen3n.pk0208.h3 | Corn |
| | p0072.comf188rb | Corn |
| | srm.pk0017.h9 | Soybean |
| | wlmk1.pk0009.b7 | Wheat |
| | wr1.pk0093.f11 | Wheat |
| Cyclin delta-1 | p0098.cdfae90r | Corn |
| | p0116.cesaf50r | Corn |
| | p0128.cpiad46rb | Corn |
| | rl0n.pk0031.e6 | Rice |
| | sah1c.pk003.i7 | Soybean |
| | sr1.pk0001.g5 | Soybeans |
| | se6.pk0028.f11 | Soybean |
| | wle1n.pk0036.e2 | Wheat |
| Cyclin delta-2 | ceb5.pk0049.h5 | Corn |
| | rl0n.pk091.m14 | Rice |
| | wr1.pk0112.b2 | Wheat |
| | wre1n.pk0104.c1 | Wheat |
| Cyclin delta-3 | cr1n.pk0185.g7 | Corn |
| | ses9c.pk002.h24 | Soybean |
| | scb1c.pk002.c13 | Soybean |
| | sr1.pk0011.d11 | Soybean |
| | sfl1.pk0001.a8 | Soybean |
| | sre.pk0035.b5 | Soybean |

The nucleic acid fragments of the instant invention may be used to isolate cDNAs and genes encoding homologous proteins from the same or other plant species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other cyclin A, cyclin delta-1, cyclin delta-2 or cyclin delta-3 proteins, either as cDNAs or genomic DNAs, could be isolated directly by using all or a portion of the instant nucleic acid fragments as DNA hybridization probes to screen libraries from any desired plant employing methodology well known to those skilled in the art. Specific oligonucleotide probes based upon the instant nucleic acid sequences can be designed and synthesized by methods known in the art (Maniatis). Moreover, the entire sequences can be used directly to synthesize DNA probes by methods known to the skilled artisan such as random primer DNA labeling, nick translation, or end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

In addition, two short segments of the instant nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the MRNA precursor encoding plant genes. Alternatively, the second primer sequence may be based upon sequences derived from the cloning vector. For example, the skilled artisan can follow the RACE protocol (Frohman et al., (1988) PNAS USA 85:8998) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al., (1989) PNAS USA 86:5673; Loh et al., (1989) Science 243:217). Products generated by the 3′ and 5′ RACE procedures can be combined to generate full-length cDNAs (Frohman, M. A. and Martin, G. R., (1989) Techniques 1:165).

Availability of the instant nucleotide and deduced amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner, R. A. (1984) Adv. Immunol. 36:1; Maniatis).

The nucleic acid fragments of the instant invention may be used to create transgenic plants in which the disclosed cyclin A, cyclin delta-1, cyclin delta-2 or cyclin delta-3 are present at higher or lower levels than normal or in cell types or developmental stages in which they are not normally found. This would have the effect of altering the regulation of cell division in those cells. The nucleic acid fragments may be used to express cyclins in plant cells to enhance cell tissue culture growth. Accordingly, the availability of nucleic acid sequences encoding all or a portion of cyclins would facilitate studies to better understand cell cycle in plants, provide genetic tools to enhance cell growth in tissue culture, increase the efficiency of gene transfer and help provide more stable transformations. Cyclins may also provide targets to facilitate design and/or identification of inhibitors of cyclins that may be useful as herbicides.

Overexpression of the cyclin A, cyclin delta-1, cyclin delta-2 or cyclin delta-3 proteins of the instant invention may be accomplished by first constructing a chimeric gene in which the coding region is operably linked to a promoter capable of directing expression of a gene in the desired tissues at the desired stage of development. For reasons of convenience, the chimeric gene may comprise promoter sequences and translation leader sequences derived from the same genes. 3′ Non-coding sequences encoding transcription termination signals may also be provided. The instant chimeric gene may also comprise one or more introns in order to facilitate gene expression.

Plasmid vectors comprising the instant chimeric gene can then constructed. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

For some applications it may be useful to direct the instant cyclin proteins to different cellular compartments, or to facilitate its secretion from the cell. It is thus envisioned that the chimeric gene described above may be further supplemented by altering the coding sequence to encode cyclin A, cyclin delta-1, cyclin delta-2 or cyclin delta-3 with appropriate intracellular targeting sequences such as transit sequences (Keegstra, K. (1989) Cell 56:247-253), signal sequences or sequences encoding endoplasmic reticulum localization (Chrispeels, J. J., (1991) Ann. Rev. Plant Phys. Plant Mol. Biol. 42:21-53), or nuclear localization signals (Raikhel, N. (1992) Plant Phys. 100:1627-1632) added and/or with targeting sequences that are already present removed. While the references cited give examples of each of these, the list is not exhaustive and more targeting signals of utility may be discovered in the future.

It may also be desirable to reduce or eliminate expression of genes encoding cyclin A, cyclin delta-1, cyclin delta-2 or cyclin delta-3 in plants for some applications. In order to accomplish this, a chimeric gene designed for co-suppression of the instant cyclin proteins can be constructed by linking a gene or gene fragment encoding an cyclin A, cyclin delta-1, cyclin delta-2 or cyclin delta-3 to plant promoter sequences. Alternatively, a chimeric gene designed to express antisense RNA for all or part of the instant nucleic acid fragment can be constructed by linking the gene or gene fragment in reverse orientation to plant promoter sequences. Either the co-suppression or antisense chimeric genes could be introduced into plants via transformation wherein expression of the corresponding endogenous genes are reduced or eliminated.

The instant cyclin A, cyclin delta-1, cyclin delta-2 or cyclin delta-3 proteins (or portions thereof) may be produced in heterologous host cells, particularly in the cells of microbial hosts, and can be used to prepare antibodies to the these proteins by methods well known to those skilled in the art. The antibodies are useful for detecting cyclin A, cyclin delta-1, cyclin delta-2 and cyclin delta-3 in situ in cells or in vitro in cell extracts. Preferred heterologous host cells for production of the instant cyclin A, cyclin delta-1, cyclin delta-2 or cyclin delta-3 are microbial hosts. Microbial expression systems and expression vectors containing regulatory sequences that direct high level expression of foreign proteins are well known to those skilled in the art. Any of these could be used to construct a chimeric gene for production of the instant cyclin A, cyclin delta-1, cyclin delta-2 or cyclin delta-3 proteins. This chimeric gene could then be introduced into appropriate microorganisms via transformation to provide high level expression of the encoded cyclin proteins. An example of a vector for high level expression of the instant cyclin A, cyclin delta-1, cyclin delta-2 or cyclin delta-3 in a bacterial host is provided (Example 9).

Additionally, the instant cyclin A, cyclin delta-1, cyclin delta-2 or cyclin delta-3 can be used as a targets to facilitate design and/or identification of inhibitors of those enzymes that may be useful as herbicides. This is desirable because the delta-1 cyclin, delta-3 cyclin, cyclin A or cyclin D described herein control various steps in the regulation of cell division. Accordingly, inhibition of the activity of one or more of the cyclins described herein could lead to inhibition plant growth. Thus, the instant delta-1 cyclin, delta-3 cyclin, cyclin A or cyclin D could be appropriate for new herbicide discovery and design.

All or a substantial portion of the nucleic acid fragments of the instant invention may also be used as probes for genetically and physically mapping the genes that they are a part of, and as markers for traits linked to those genes. Such information may be useful in plant breeding in order to develop lines with desired phenotypes. For example, the instant nucleic acid fragments may be used as restriction fragment length polymorphism (RFLP) markers. Southern blots (Maniatis) of restriction-digested plant genomic DNA may be probed with the nucleic acid fragments of the instant invention. The resulting banding patterns may then be subjected to genetic analyses using computer programs such as MapMaker (Lander et at., (1987) Genomics 1:174-181) in order to construct a genetic map. In addition, the nucleic acid fragments of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein, D. et al., (1980) Am. J. Hum. Genet. 32:314-331).

The production and use of plant gene-derived probes for use in genetic mapping is described in R. Bernatzky, R. and Tanksley, S. D. (1986) Plant Mol. Biol. Reporter 4(1):37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequences may also be used for physical mapping (i.e., placement of sequences on physical maps; see Hoheisel, J. D., et al., In: Nonmammalian Genomic Analysis: A Practical Guide, Academic press 1996, pp. 319-346, and references cited therein).

In another embodiment, nucleic acid probes derived from the instant nucleic acid sequences may be used in direct fluorescence in situ hybridization (FISH) mapping (Trask, B. J. (1991) Trends Genet. 7:149-154). Although current methods of FISH mapping favor use of large clones (several to several hundred KB; see Laan, M. et al. (1995) Genome Research 5:13-20), improvements in sensitivity may allow performance of FISH mapping using shorter probes.

A variety of nucleic acid amplification-based methods of genetic and physical mapping may be carried out using the instant nucleic acid sequences. Examples include allele-specific amplification (Kazazian, H. H. (1989) J. Lab. Clin. Med. 114(2):95-96), polymorphism of PCR-amplified fragments (CAPS; Sheffield, V. C. et al. (1993) Genomics 16:325-332), allele-specific ligation (Landegren, U. et al. (1988) Science 241:1077-1080), nucleotide extension reactions (Sokolov, B. P. (1990) Nucleic Acid Res. 18:3671), Radiation Hybrid Mapping (Walter, M. A. et al. (1997) Nature Genetics 7:22-28) and Happy Mapping (Dear, P. H. and Cook, P. R. (1989) Nucleic Acid Res. 17:6795-6807). For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Loss of function mutant phenotypes may be identified for the instant cDNA clones either by targeted gene disruption protocols or by identifying specific mutants for these genes contained in a maize population carrying mutations in all possible genes (Ballinger and Benzer, (1989) Proc. Natl. Acad. Sci USA 86:9402; Koes et al., (1995) Proc. Natl. Acad. Sci USA 92:8149; Bensen et al., (1995) Plant Cell 7:75). The latter approach may be accomplished in two ways. First, short segments of the instant nucleic acid fragments may be used in polymerase chain reaction protocols in conjunction with a mutation tag sequence primer on DNAs prepared from a population of plants in which Mutator transposons or some other mutation-causing DNA element has been introduced (see Bensen, supra). The amplification of a specific DNA fragment with these primers indicates the insertion of the mutation tag element in or near the plant gene encoding the cyclin A, cyclin delta-1, cyclin delta-2 or cyclin delta-3. Alternatively, the instant nucleic acid fragment may be used as a hybridization probe against PCR amplification products generated from the mutation population using the mutation tag sequence primer in conjunction with an arbitrary genomic site primer, such as that for a restriction enzyme site-anchored synthetic adaptor. With either method, a plant containing a mutation in the endogenous gene encoding a cyclin A, cyclin delta-1, cyclin delta-2 or cyclin delta-3 can be identified and obtained. This mutant plant can then be used to determine or confirm the natural function of the cyclin A, cyclin delta-1, cyclin delta-2 or cyclin delta-3 gene product.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

### Composition of cDNA Libraries; Isolation and Sequencing of cDNA Clones

cDNA libraries representing mRNAs from various corn, rice, soybean and wheat tissues were prepared. The characteristics of the libraries are described below.

TABLE 2
cDNA Libraries from Corn, Rice, Soybean and Wheat

| Library | Tissue | Clone |
|---|---|---|
| ceb5 | Corn embryo 30 days after pollination | ceb5.pk0049.h5 |
| cen3n | Corn endosperm 20 days After pollination* | cen3n.pk0208.h3 |
| cr1n | Corn root from 7 day old seedlings* | cr1n.pk0185.g7 |
| p0072 | Corn mesocotyl: 14 days after planting etiolated seedling | p0072.comf188rb |
| p0098 | Corn ear shoot, prophasei (2.8-4.8 cm)* | p0098.cdfae90r |
| p0116 | Corn, DAM methylase induced transgenic suspension cells** | p0116.cesaf50r |
| p0128 | Corn primary and secondary immature ear tissue pooled | p0128.cpiad46rb |
| rl0n | Rice 15 day old leaf* | rl0n.pk0031.e6 |
| | | rl0n.pk091.m14 |
| sah1c | Soybean sprayed with Authority ™ herbicide | sah1c.pk003.i7 |
| scb1c | Soybean embryogenic suspension culture | scb1c.pk002.c13 |
| se6 | Soybean embryo, 26 days after flowering | se6.pk0028.f11 |
| ses9c | Soybean embryogenic suspension | ses9c.pk002.h24 |
| sfl1 | Soybean immature flower | sfl1.pk0001.a8 |
| sr1 | Soybean root | sr1.pk0001.g5 |
| | | sr1.pk0011.d11 |
| sre | Soybean root elongation | sre.pk0035.b5 |
| srm | Soybean root meristem | srm.pk0017.h9 |
| wle1n | Wheat leaf; 7 day old etiolated seedling* | wle1n.pk0036.e2 |
| wlmk1 | Wheat seedlings 1 hr after inoculation with Erysiphe | wlmk1.pk0009.b7 |
| | graminis f. sp tritici and treatment with fungicide*** | |
| wr1 | Wheat root; 7 day old seedling, light grown | wr1.pk0093.f11 |
| | | wr1.pk0112.b2 |
| wre1n | Wheat root; 7 day old etiolated seedling* | wre1n.pk0104.c1 |

*These libraries were normalized essentially as described in U.S. Pat. No. 5,482,845
**cell line is transgenic for a vector harboring four copies of the estrogen response element (ERE) and CaMV-59 promoter driving dam methylase expression (Klein-Hitpab, L., et al., (1989) Cell 46: 1053-1061). Expression of dam methylase was induced by 17 alpha-ethnylestradiol. Library was also normalized as described above
***Application of 6-iodo-2-propoxy-3-propyl-4(3H)-quinazolinone; synthesis and methods of using this compound are described in USSN 08/545,827, incorporated herein by reference.

cDNA libraries were prepared in Uni-ZAP™ XR vectors according to the manufacturer's protocol (Stratagene Cloning Systems, La Jolla, Calif.). Conversion of the Uni-ZAP™ XR libraries into plasmid libraries was accomplished according to the protocol provided by Stratagene. Upon conversion, cDNA inserts were contained in the plasmid vector pBluescript. cDNA inserts from randomly picked bacterial colonies containing recombinant pBluescript plasmids were amplified via polymerase chain reaction using primers specific for vector sequences flanking the inserted cDNA sequences or plasmid DNA was prepared from cultured bacterial cells. Amplified insert DNAs or plasmid DNAs were sequenced in dye-primer sequencing reactions to generate partial cDNA sequences (expressed sequence tags or “ESTs”; see Adams, M. D. et al., (1991) Science 252:1651). The resulting ESTs were analyzed using a Perkin Elmer Model 377 fluorescent sequencer.

### Example 2

### Identification of cDNA Clones

ESTs encoding cyclin proteins were identified by conducting BLAST (Basic Local Alignment Search Tool; Altschul, S. F., et al., (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D. J. (1993) Nature Genetics 3:266-272 and Altschul, Stephen F., et al. (1997) Nucleic Acids Res. 25:3389-3402) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

### Example 3

### Characterization of cDNA Clones Encoding Cyclin A Proteins

The BLASTX search using the EST sequences from clones cen3n.pk0208.h3 and p0072.comf188rb revealed similarity of the proteins encoded by the cDNAs to cyclin A from Catharanthus roseus (NCBI Identifier No. gi 2190259). The BLASTX search using the EST sequences from clones srm.pk0017.h9 revealed similarity of the protein encoded by the cDNA to cyclin A from Glycine max (NCBI Identifier No. gi 857393). The BLASTX search using the EST sequences from clones w1nk1.pk0009.b7 and wrl.pk0093.f11 revealed similarity of the proteins encoded by the cDNAs to cyclin A from Glycine max (NCBI Identifier No. gi 857395).

In the process of comparing the ESTs it was found that corn clones cen3n.pk0208.h3 and p0072.comf188rb had overlapping regions of homology. Wheat clones w1mk1.pk0009.b7 and wr1.pk0093.f11 were also found to have overlapping regions of homology. Using this homology it was possible to align the ESTs and assemble two individule contigs encoding unique corn and wheat cyclin A proteins.

The BLAST results for each of the contigs and the soybean EST are shown in Table 3:

TABLE 3
BLAST Results for Clones Encoding Polypeptides Homologous
to Catharanthus roseus and Glycine max Cyclin A Proteins

| Clone | BLAST pLog Score |
|---|---|
| Contig Composed of: | 124.00 |
| cen3n.pk0208.h3 | |
| p0072.comf188rb | |
| srm.pk0017.h9 | 29.22 |
| Contig Composed of: | 72.04 |
| wlmk1.pk0009.b7 | |
| wr1.pk0093.f11 | |

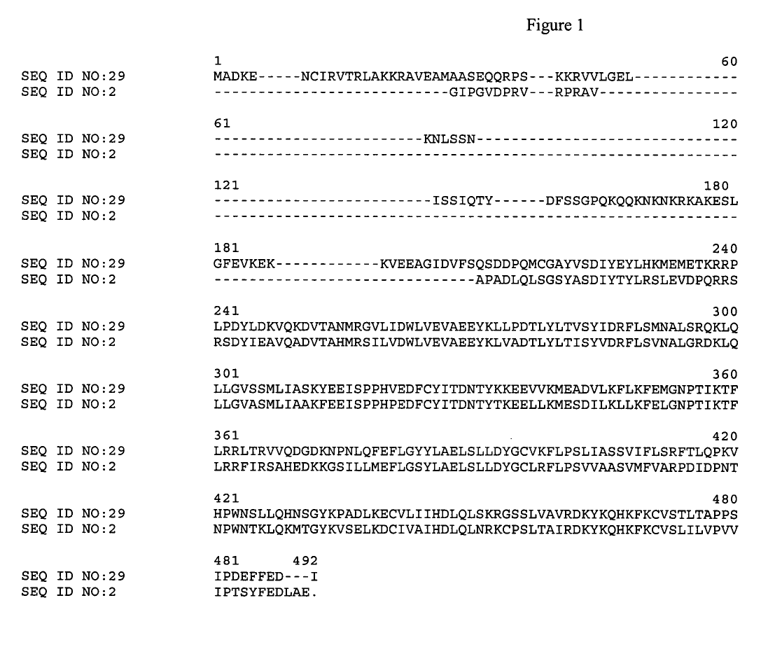

The sequence of the corn contig composed of clones cen3n.pk0208.h3 and p0072.comf188rb is shown in SEQ ID NO:1; the deduced amino acid sequence of this contig, which represents 80% of the protein (the C-terminal region), is shown in SEQ ID NO:2. The amino acid sequence set forth in SEQ ID NO:2 was evaluated by BLASTP, yielding a pLog value of 105.00 versus the Catharanthus roseus sequence. FIG. 1 presents an alignment of the amino acid sequence set forth in SEQ ID NO:2 and the Catharanthus roseus sequence. A calculation of the percent similarity of the amino acid sequence set forth in SEQ ID NO:2 and the Catharanthus roseus sequence revealed that SEQ ID NO:2 was 60% similar to the Catharanthus roseus sequence. Sequence alignments and percent similarity calculations were performed by the Clustal Algorithm (Higgins, D. G. et al., (1989) CABIOS 5(2):151-153), using the Megalign program of the LASARGENE bioinformatics computing suite (DNASTAR Inc., Madison, Wis.) (hereinafter “Clustal Algorithm”). Default parameters for the Clustal method for protein multiple alignments were: GAP PENALTY=10, GAP LENGTH PENALTY=10; for pairwise alignments KTUPLE 1, GAP PENALTY=3, WINDOW=5 and DIAGONALS SAVED=5.

The sequence of a portion of the cDNA insert from clone srm.pk0017.h9 is shown in SEQ ID NO:3; the deduced amino acid sequence of this cDNA, which represents 22% of the protein (the N-terminal region), is shown in SEQ ID NO:4. The amino acid sequence of clone srm.pk0017.h9 appears to represent a new soybean cyclin A protein due to the fact that it was only 16% similar (as calculated by the Clustal Algorithm) to a cyclin A from Glycine max (NCBI Identifier No. gi 857393).

The sequence of the wheat contig composed of clones w1mk1.pk0009.b7 and wr1.pk0093.f11 is shown in SEQ ID NO:5; the deduced amino acid sequence of this contig, which represents 42% of the protein (the C-terminal region), is shown in SEQ ID NO:6. A calculation of the percent similarity of the amino acid sequence set forth in SEQ ID NO:6 and the Glycine max (NCBI Identifier No. gi 857395) sequence (using the Clustal Algorithm) revealed that SEQ ID NO:6 was 54% similar to the soybean cyclin A sequence.

BLAST scores and probabilities indicate that the instant nucleic acid fragments encode portions of cyclin A proteins. These sequences represent the first corn, rice and wheat sequences and a new soybean sequence encoding cyclin A proteins.

### Example 4

### Characterization of cDNA Clones Encoding Cyclin Delta-1 Proteins

The BLASTX search using the nucleotide sequences from clones p0098.cdfae90r, p0116.cesaf50r, p0128.cpiad46rb, r10n.pk0031.e6, sah1c.pk003.i7, sr1.pk0001.g5 and se6.pk0028.f11 revealed similarity of the proteins encoded by the cDNAs to cyclin delta-1 from Arabidopsis thaliana (NCBI Identifier No. gi 3915635). The BLASTX search using the nucleotide sequences from clone w1e1n.pk0036.e2 revealed similarity of the protein encoded by the cDNA to cyclin 1a from Zea mays (NCBI Identifier No. gi 2130119).

In the process of comparing the ESTs it was found that corn clones p0098.cdfae9or, p0116.cesaf50r and p0128.cpiad46rb had overlapping regions of homology. Soybean clones sah1c.pk003.i7 and sr1.pk0001.g5 were also found to have overlapping regions of homology. Using this homology it was possible to align the ESTs and assemble two individule contigs encoding unique corn and soybean cyclin delta-1 proteins.

The BLAST results for each of the contigs and other ESTs are shown in Table 4:

TABLE 4
BLAST Results for Clones Encoding Polypeptides Homologous
to Arabidopsis thaliana and Zea mays Cyclin Delta-1 Proteins

| Clone | BLAST pLog Score |
|---|---|
| Contig Composed of: | 33.22 |
| p0098.cdfae90r | |
| p0116.cesaf50r | |
| p0128.cpiad46rb | |
| rl0n.pk0031.e6 | 22.40 |
| Contig Composed of: | 115.00 |
| sah1c.pk003.i7 | |
| sr1.pk0001.g5 | |
| se6.pk0028.f11 | 112.00 |
| wle1n.pk0036.e2 | 30.70 |

The sequence of the corn contig composed of clones p0098.cdfae90r, p0116.cesaf50r and p0128.cpiad46rb is shown in SEQ ID NO:7; the deduced amino acid sequence of this contig, which represents 71 % of the protein (the N-terminal region), is shown in SEQ ID NO:8. The amino acid sequence set forth in SEQ ID NO:8 was evaluated by BLASTP, yielding a pLog value of 30.52 versus the A. thaliana sequence. SEQ ID NO:8 is only 18% similar (as calculated by the Clustal Algorithm) to cyclin delta-1 from Zea mays (NCBI Identifier No. gi 2130119) which suggests that SEQ ID NO:8 represents a new corn cyclin delta-1 protein.

A portion of the sequence of the cDNA insert in clone r10n.pk0031.e6 was determined and is shown in SEQ ID NO:9; the deduced amino acid sequence of this cDNA, which represents 53% of the protein (the N-terminal region), is shown in SEQ ID NO:10. The amino acid sequence set forth in SEQ ID NO:10 was evaluated by BLASTP, yielding a pLog value of 22.00 versus the A. thaliana sequence.

The sequence of the soybean contig composed of clones sahlc.pk003.i7 and sr1.pk0001.g5 is shown in SEQ ID NO:11; the deduced amino acid sequence of this contig, which represents 100% of the protein, is shown in SEQ ID NO:12. The amino acid sequence set forth in SEQ ID NO:12 was evaluated by BLASTP, yielding a pLog value of 108.00 versus the A. thaliana sequence.

The entire sequence of the cDNA insert in clone se6.pk0028.f11 was determined and is shown in SEQ ID NO:13; the deduced amino acid sequence of this cDNA, which represents 95% of the protein, is shown in SEQ ID NO:14. The amino acid sequence set forth in SEQ ID NO:14 was evaluated by BLASTP, yielding a pLog value of 103.00 versus the A. thaliana sequence.

The sequence of a portion of the cDNA insert from clone w1e1n.pk0036.e2 is shown in SEQ ID NO:15; the deduced amino acid sequence of this cDNA is shown in SEQ ID NO:16.

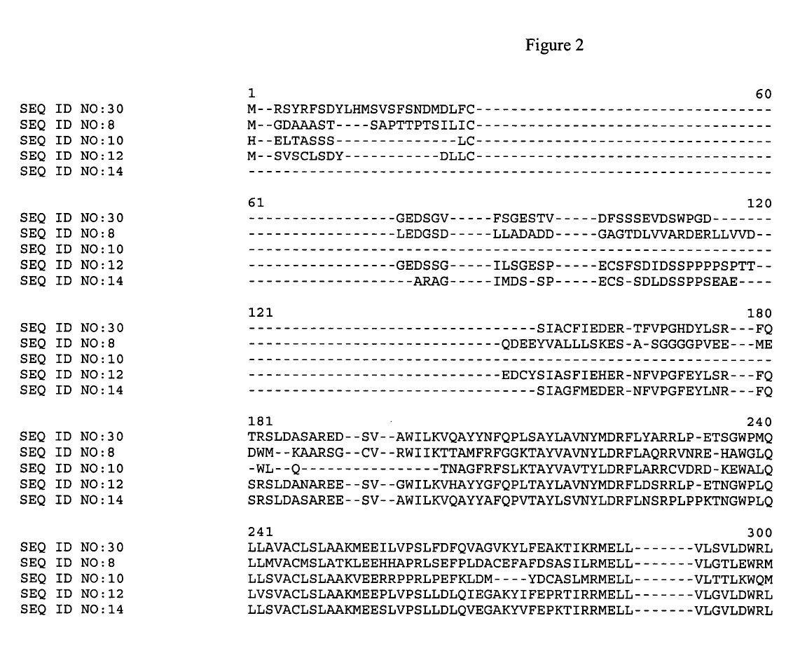

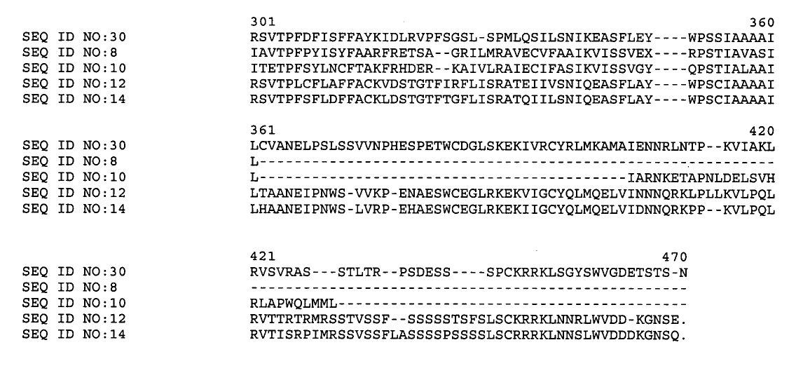

FIG. 2 presents an alignment of the amino acid sequences set forth in SEQ ID NOs:8, 10,12 and 14 and the A. thaliana cyclin delta-1 sequence. The data in Table 5 represents a calculation of the percent similarity of the amino acid sequences set forth in SEQ ID NOs:8, 10, 12 and 14 and the A. thaliana cyclin delta-1 sequence.

TABLE 5
Percent Identity of Amino Acid Sequences Deduced From the
Nucleotide Sequences of cDNA Clones Encoding Polypeptides
Homologous to Arabidopsis thaliana Cyclin Delta-1 Proteins

| Clone | SEQ ID NO. | Percent Similarity |
|---|---|---|
| Contig Composed of: | 8 | 29% |
| p0098.cdfae90r | | |
| p0116.cesaf50r | | |
| p0128.cpiad46rb | | |
| rl0n.pk0031.e6 | 10 | 31% |
| Contig Composed of: | 12 | 54% |
| sah1c.pk003.i7 | | |
| sr1.pk0001.g5 | | |
| se6.pk0028.f11 | 14 | 54% |

Sequence alignments and sequence percent similarity calculations were performed by the Clustal Algorithm. Sequence alignments, BLAST scores and probailities indicate that the instant nucleic acid fragments encode entire, nearly entire or portions of cyclin delta-1 proteins. These sequences represent the first rice, soybean and wheat sequences and a new corn sequence encoding cyclin delta-1 proteins.

### Example 5

### Characterization of cDNA Clones Encoding Cyclin Delta-2 Proteins

The BLASTX search using the nucleotide sequences from clones ceb5.pk0049.h5, r10n.pk091.m14, and wre1n.pk0104.c1 revealed similarity of the proteins encoded by the cDNAs to cyclin delta-2 from Nicotina tabacum (NCBI Identifier No. gi 4160298). The BLAST results for each of these ESTs are shown in Table 6:

TABLE 6
BLAST Results for Clones Encoding Polypeptides Homologous
to Nicotina tabacum Cyclin Delta-2 Proteins

| Clone | BLAST pLog Score |
|---|---|
| ceb5.pk0049.h5 | 65.22 |
| rl0n.pk091.m14 | 9.10 |
| wre1n.pk0104.c1 | 19.70 |

The sequence of the entire cDNA insert in clone ceb5.pk0049.h5 was determined and is shown in SEQ ID NO:17; the deduced amino acid sequence of this cDNA, which represents 94% of the protein, is shown in SEQ ID NO:18. The amino acid sequence set forth in SEQ ID NO:18 was evaluated by BLASTP, yielding a pLog value of 64.10 versus the Nicotina tabacum sequence.

The sequence of a portion of the cDNA insert from clone r10n.pk091.ml4 is shown in SEQ ID NO:19; the deduced amino acid sequence of this cDNA, which represents 30% of a cyclin delta-2 protein (the C-terminal region), is shown in SEQ ID NO:20.

The sequence of the entire cDNA insert in clone wre1n.pk0104.c1 was determined and is shown in SEQ ID NO:21; the deduced amino acid sequence of this cDNA, which represents 26% of the protein (the C-terminal region), is shown in SEQ ID NO:22. The amino acid sequence set forth in SEQ ID NO:22 was evaluated by BLASTP, yielding a pLog value of 13.00 versus the Nicotina tabacum sequence.

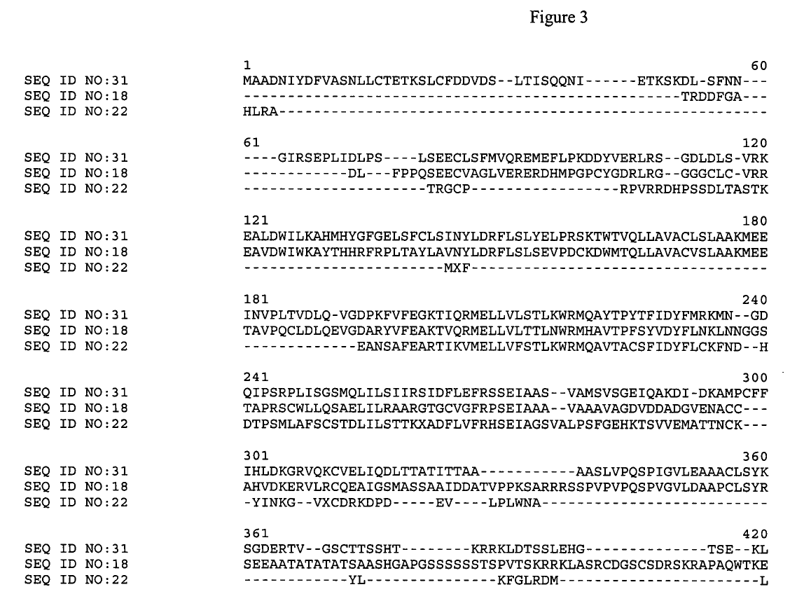

FIG. 3 presents an alignment of the amino acid sequences set forth in SEQ ID NOs:18 and 22 and the Nicotina tabacum sequence. The data in Table 7 represents a calculation of the percent similarity of the amino acid sequences set forth in SEQ ID NOs:18 22 and the Nicotina tabacum sequence.

TABLE 7
Percent Identity of Amino Acid Sequences Deduced From the
Nucleotide Sequences of cDNA Clones Encoding Polypeptides
Homologous to Nicotina tabacum Cyclin Delta-2 Proteins

| Clone | SEQ ID NO. | Percent Similarity |
|---|---|---|
| ceb5.pk0049.h5 | 18 | 37% |
| wre1n.pk0104.c1 | 22 | 32.5% |

Sequence alignments and sequence percent similarity calculations were performed by the Clustal Algorithm. Sequence alignments and BLAST scores and probabilities indicate that the instant nucleic acid fragments encode entire, nearly entire or portions of cyclin delta-2 proteins. These sequences represent the first corn, rice and wheat sequences encoding cyclin delta-2 proteins.

### Example 6

### Characterization of cDNA Clones Encoding Cyclin Delta-3 Proteins

The BLASTX search using the nucleotide sequences from clones cr1n.pk0185.g7, ses9c.pk002.h24, scb1c.pk002.c13 and sr1.pk0011.d11 revealed similarity of the proteins encoded by the cDNAs to cyclin delta-3 from Nicotiana tabacum (NCBI Identifier No. gi 4160300). The BLASTX search using the nucleotide sequences from clones sf11.pk0001.a8 and sre.pk0035.b5 revealed similarity of the proteins encoded by the cDNAs to cyclin delta-3 from Pisum sativum (NCBI Identifier No. gi 3608179).

In the process of comparing the ESTs it was found that soybean clones ses9c.pk002.h24, scb1c.pk002.c13 and sr1.pk0001.d11 had overlapping regions of homology. Soybean clones sf11.pk0001.a8 and sre.pk0035.b5 were also found to have overlapping regions of homology. Using this homology it was possible to align the ESTs and assemble two contigs encoding unique soybean cyclin delta-3 proteins.

The BLAST results for each of these contigs and the corn EST are shown in Table 8:

TABLE 8
BLAST Results for Clones Encoding Polypeptides Homologous
to Nicotiana tabacum and Pisum sativum Cyclin Delta-3 Proteins

| Clone | BLAST pLog Score |
|---|---|
| cr1n.pk0185.g7 | 35.30 |
| Contig Composed of: | 29.22 |
| ses9c.pk002.h24 | |
| scb1c.pk002.c13 | |
| sr1.pk0011.d11 | |
| Contig Composed of: | 18.00 |
| sfl1.pk0001.a8 | |
| sre.pk0035.b5 | |

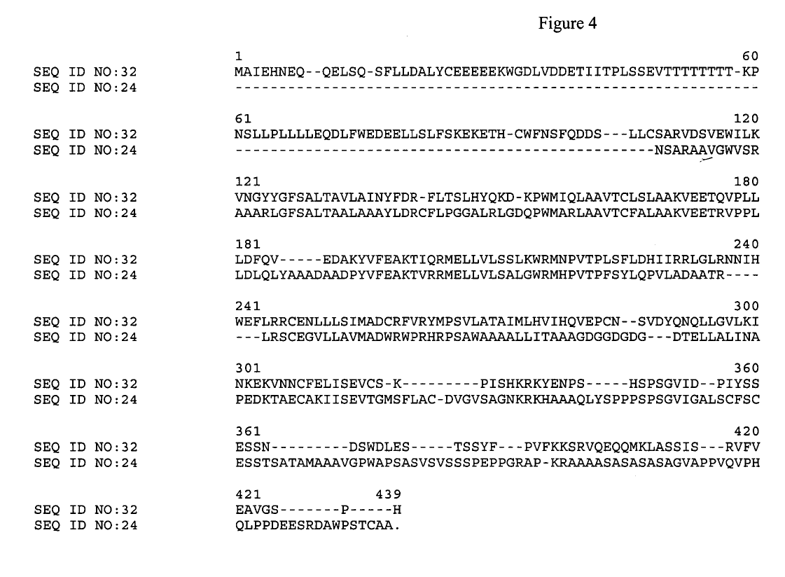

A large portion of the cDNA insert in clone cr1n.pk0185.g7 was determined and is shown in SEQ ID NO:23; the deduced amino acid sequence of this cDNA, which represents 85% of the protein (C-terminal region) is shown in SEQ ID NO:24. The amino acid sequence set forth in SEQ ID NO:24 was evaluated by BLASTP, yielding a pLog value of 38.10 versus the Nicotiana tabacum sequence. A calculation (using the Clustal Algorithm) of the percent similarity of the amino acid sequence set forth in SEQ ID NO:24 and the Nicotiana tabacum sequence revealed SEQ ID NO:24 was 27% to the Nicotiana tabacum sequence. FIG. 4 presents an alignment of the amino acid sequences set forth in SEQ ID NO:24 and the Nicotiana tabacum sequence.

The sequence of the soybean contig composed of clones ses9c.pk002.h24, scb1c.pk002.c13 and sr1.pk0011.d11 is shown in SEQ ID NO:25; the deduced amino acid sequence of this contig, which represents 42% of the protein (the N-terminal region) is shown in SEQ ID NO:26.

The sequence of the soybean contig composed of clones sf11.pk0001.a8 and sre.pk0035.b5 is shown in SEQ ID NO:27; the deduced amino acid sequence of this contig, which represents 21% of the protein (N-terminal region) is shown in SEQ ID NO:28.

Sequence alignments and BLAST scores and probabilities indicate that the instant nucleic acid fragments encode entire, nearly entire or portions of cyclin delta-3 proteins. These sequences represent the first corn and soybean sequences encoding cyclin delta-3 proteins.

### Example 7

### Expression of Chimeric Genes in Monocot Cells

A chimeric gene comprising a cDNA encoding a cyclin protein in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (NcoI or Smal) can be incorporated into the oligonucleotides to provide proper orientation of the DNA fragment when inserted into the digested vector pML103 as described below. Amplification is then performed in a standard PCR. The amplified DNA is then digested with restriction enzymes NcoI and SmaI and fractionated on an agarose gel. The appropriate band can be isolated from the gel and combined with a 4.9 kb NcoI-SmaI fragment of the plasmid pML103. Plasmid pML103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C overnight, essentially as described (Maniatis). The ligated DNA may then be used to transform E. coli XL1-Blue (Epicurian Coli XL-1 Blue™; Stratagene). Bacterial transformants can be screened by restriction enzyme digestion of plasmid DNA and limited nucleotide sequence analysis using the dideoxy chain termination method (Sequenase™ DNA Sequencing Kit; U.S. Biochemical). The resulting plasmid construct would comprise a chimeric gene encoding, in the 5′ to 3′ direction, the maize 27 kD zein promoter, a cDNA fragment encoding a cyclin protein, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al., (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al., (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 μg per niL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules CA), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al., (1990) Bio/Technology 8:833-839).

### Example 8

### Expression of Chimeric Genes in Dicot Cells

A seed-specific expression cassette composed of the promoter and transcription terminator from the gene encoding the P subunit of the seed storage protein phaseolin from the bean Phaseolus vulgaris (Doyle et al. (1986) J. Biol. Chem. 261:9228-9238) can be used for expression of the instant cyclin proteins in transformed soybean. The phaseolin cassette includes about 500 nucleotides upstream (5′ ) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), Sma I, Kpn I and Xba I. The entire cassette is flanked by Hind III sites.

The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC18 vector carrying the seed expression cassette.

Soybean embroys may then be transformed with the expression vector comprising sequences encoding cyclin proteins. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Kline et al. (1987) Nature (London) 327:70, U.S. Pat. No. 4,945,050). A DuPont BioliStic™ PDS 1000/HE instrument (helium retrofit) can be used for these transformations.

A selectable marker gene which can be used to facilitate soybean transformation is a chimeric gene composed of the 35S promoter from Cauliflower Mosaic Virus (Odell et al. (1985) Nature 313:810-812), the hygromycin phosphotransferase gene from plasmid pJR225 (from E. coli; Gritz et al.(1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the cyclin protein and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 pg/gL), 20 μl spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 9

### Expression of Chimeric Genes in Microbial Cells

The cDNAs encoding the instant cyclin proteins can be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% NuSieve GTG™ low melting agarose gel (FMC). Buffer and agarose contain 10 pg/ml ethidium bromide for visualization of the DNA fragment. The fragment can then be purified from the agarose gel by digestion with GELase™ (Epicentre Technologies) according to the manufacturer's instructions, ethanol precipitated, dried and resuspended in 20 μL of water. Appropriate oligonucleotide adapters may be ligated to the fragment using T4 DNA ligase (New England Biolabs, Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as decribed above. The prepared vector pBT430 and fragment can then be ligated at 16° C. for 15 hours followed by transformation into DH5 electrocompetent cells (GIBCO BRL). Transformants can be selected on agar plates containing LB media and 100 pg/mL ampicillin. Transformants containing the gene encoding the cyclin protein are then screened for the correct orientation with respect to the T7 promoter by restriction enzyme analysis.

For high level expression, a plasmid clone with the cDNA insert in the correct orientation relative to the T7 promoter can be transformed into E. coli strain BL2 1 (DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 h at 25°. Cells are then harvested by centrifugation and re-suspended in 50 μL of 50 mM Tris-HC1 at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One μg of protein from the soluble fraction of the culture can be separated by SDS-polyacrylamide gel electrophoresis. Gels can be observed for protein bands migrating at the expected molecular weight.

### Example 10

### Evaluating Compounds for Their Ability to Inhibit the Activity of Cyclin Proteins

The cyclin proteins described herein may be produced using any number of methods known to those skilled in the art. Such methods include, but are not limited to, expression in bacteria as described in Example 9, or expression in eukaryotic cell culture, in planta, and using viral expression systems in suitably infected organisms or cell lines. The instant cyclin proteins may be expressed either as mature forms of the proteins as observed in vivo or as fusion proteins by covalent attachment to a variety of enzymes, proteins or affinity tags. Common fusion protein partners include glutathione S-transferase (“GST”), thioredoxin (“Trx”), maltose binding protein, and C- and/or N-terminal hexahistidine polypeptide (“(His)₆”). The fusion proteins may be engineered with a protease recognition site at the fusion point so that fusion partners can be separated by protease digestion to yield intact mature enzyme. Examples of such proteases include thrombin, enterokinase and factor Xa. However, any protease can be used which specifically cleaves the peptide connecting the fusion protein and the enzyme.

Purification of the instant cyclin proteins, if desired, may utilize any number of separation technologies familiar to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography and affinity chromatography, wherein the affinity ligand represents a substrate, substrate analog or inhibitor. When the cyclin proteins are expressed as fusion proteins, the purification protocol may include the use of an affinity resin which is specific for the fusion protein tag attached to the expressed enzyme or an affinity resin containing ligands which are specific for the enzyme. For example, a cyclin protein may be expressed as a fusion protein coupled to the C-terminus of thioredoxin. In addition, a (His)₆ peptide may be engineered into the N-terminus of the fused thioredoxin moiety to afford additional opportunities for affinity purification. Other suitable affinity resins could be synthesized by linking the appropriate ligands to any suitable resin such as Sepharose-4B. In an alternate embodiment, a thioredoxin fusion protein may be eluted using dithiothreitol; however, elution may be accomplished using other reagents which interact to displace the thioredoxin from the resin. These reagents include β-mercaptoethanol or other reduced thiol. The eluted fusion protein may be subjected to further purification by traditional means as stated above, if desired. Proteolytic cleavage of the thioredoxin fusion protein and the enzyme may be accomplished after the fusion protein is purified or while the protein is still bound to the ThioBond™ affinity resin or other resin.

Crude, partially purified or purified enzyme, either alone or as a fusion protein, may be utilized in assays for the evaluation of compounds for their ability to inhibit enzymatic activition of the cyclin proteins disclosed herein. Assays may be conducted under well known experimental conditions which permit optimal enzymatic activity. For example, assays for cyclin A are presented by Kouchi, H., et al., (1995) Plant Cell 7(8):143-1155. Assays for cyclin delta-1 and cyclin delta-2 are presented by Soni B., et al., (1995) Plant Cell 7(1):85-103. Assays for cyclin delta-3 are presented by Sorrell D. A., et al., (1999) Plant Physiol. 199:343-351.

1-26. (canceled)

27. an isolated polynucleotide comprising:
(a) a nucleotide sequence encoding a polypeptide having cyclin delta activity, wherein the polypeptide has an amino acid sequence of at least 90% sequence identity, based on the Clustal method of alignment with pairwise alignment default parameters of KTUPLE=1, GAP PENALTY=3, WINDOW=5 and DIAGONALS SAVED=5, when compared to SEQ ID NO:18, or
(b) the complement of the nucleotide sequence of (a).

28. The polynucleotide of claim 27, wherein the amino acid sequence of the polypeptide has at least 95% sequence identity, based on the Clustal method of alignment with said pairwise alignment default parameters, when compared to SEQ ID NO:18.

29. The polynucleotide of claim 27, wherein the amino acid sequence of the polypeptide comprises SEQ ID NO:18.

30. The polynucleotide of claim 27 wherein the nucleotide sequence comprises SEQ ID NO:17.

31. A vector comprising the polynucleotide of claim 27.

32. A recombinant DNA construct comprising the polynucleotide of claim 27 operably linked to at least one regulatory sequence.

33. A method for transforming a cell, comprising transforming a cell with the polynucleotide of claim 27.

34. A cell comprising the recombinant DNA construct of claim 32.

35. A plant comprising the recombinant DNA construct of claim 32.

36. A seed comprising the recombinant DNA construct of claim 32.
